(19) United States
(12) Patent Application Publication
(10) Pub. No.: US 2025/0061995 A1
(43) Pub. Date: Feb. 20, 2025

(54) Couple-Based Digital Reproductive Health Platform System and Method

(71) Applicant: Vie Science, Inc., Brookline, MA (US)

(72) Inventor: Carmen Messerlian, Brookline, MA (US)

(73) Assignee: Vie Science, Inc., Brookline, MA (US)

(21) Appl. No.: 18/807,292

(22) Filed: Aug. 16, 2024

Related U.S. Application Data
(60) Provisional application No. 63/533,226, filed on Aug. 17, 2023.

(51) Int. Cl. G16H 20/00; G16H 10/20

(52) U.S. Cl. CPC G16H 20/00; G16H 10/20

(57) ABSTRACT

A reproductive health platform system. The system may include a server, the server including a reproductive health application, ML algorithms, multimodal AI, reproductive health GUI, controller, operating memory, a communications interface. The system may further include a data store. The controller may be configured to execute stored program instructions including collecting data related to a subject couple; analyzing the collected data; creating a digital copy of the subject couple based on the analyzed collected data; generating personalized interventions and/or predictive outcomes based on the analyzed collected data; implementing the generated personalized interventions and/or predictive outcomes in the digital copy of the subject couple; presenting personalized interventions and/or predictive outcomes to the subject couple based on results from implementing the generated personalized interventions and/or predictive outcomes in the digital copy of the subject couple; and tracking results data of the presented personalized interventions and/or predictive outcomes implemented by the subject couple.

### RELATED APPLICATIONS

This application is related and claims priority to U.S. Provisional Patent Application No. 63/533,226, filed on Aug. 17, 2023 the application of which is incorporate herein by reference in its entirety.

### TECHNICAL FIELD

The subject matter relates generally to reproductive healthcare and more particularly to a couple-based digital health platform system and methods for seamless fertility solutions.

### BACKGROUND

Although artificial intelligence (AI) has already transformed several domains of human life, its greatest impact in medicine has yet to be fully realized. Broadly speaking, AI can be defined as the synthesizing and processing of large, complex data to draw inferences from for real-world simulated applications. By mimicking human reasoning, AI predicts and selects the best decision to optimize a specific outcome. Even though it was introduced in the 20th century, AI was not applied to medical settings until more recently. In hospital and clinical environments, AI is primarily used to capture patterns in electronic health records (EHR), laboratory tests, and imaging to facilitate diagnosis, predict patient outcomes, and personalize treatment. AI also assists with drug discovery and development, EHR transcription, and remote patient care (e.g., telemedicine). Although several specialties have integrated AI into their clinical practice, its application remains limited in the setting of medically assisted reproduction (MAR) and related fertility care, with its primary application being to optimize aspects of assisted reproductive technologies (ART). For example, various machine learning algorithms have been proposed to help select the best quality sperm, egg, and embryo for implantation in the hopes of improving in-vitro fertilization (IVF) outcomes.

Although ART facilitates reproduction, the success (live birth) rate of ART per cycle remains low (≤30%) and declines with age. Further, ART only captures people seeking fertility treatment, whereas most live births among the general population result from natural conceptions. Consequently, for most pregnancies, information on potentially influential exposures and activities or experiences is not collected until after a mother-to-be attends a prenatal clinic (typically at 6 to 10 gestational weeks). Thus, the current care system misses the preconception window, which continues to be an overlooked period for data capture, assessment, and intervention.

Accordingly, because human reproduction is a complex system that requires two main counterparts (sperm and oocyte), there remains several challenges when investigating and treating infertility and reproductive health conditions using traditional analytical tools and design methods.

### SUMMARY

In one embodiment, a system for couple-based digital reproductive health platform is provided. The system may include a server. The server may include a reproductive health application, ML algorithms, multimodal AI, reproductive health GUI, controller, operating memory, and a communications interface, and wherein the server is accessible via a network. The system may further include a data store in communication with the server. The controller may be configured to execute stored program instructions, which may include collecting data related to a subject couple; analyzing the collected data related to the subject couple; creating a digital copy of the subject couple based on the analyzed collected data; generating personalized interventions and/or predictive outcomes based on the analyzed collected data related to the subject couple; implementing the generated personalized interventions and/or predictive outcomes in the digital copy of the subject couple; presenting personalized interventions and/or predictive outcomes to the subject couple based on results from implementing the generated personalized interventions and/or predictive outcomes in the digital copy of the subject couple; and tracking results data of the presented personalized interventions and/or predictive outcomes implemented by the subject couple. The data related to the subject couple may be collected via data modalities, and wherein the data modalities may include exposome data and biological data. The biological data may include omics data. presented personalized interventions and/or predictive outcomes may include the generated personalized interventions and/or predictive outcomes that resulted in a positive outcome when implemented in the digital copy of the subject couple. The multimodal Al and/or ML algorithms may process the collected data related to the subject couple, create the digital copy of the subject couple, generate the personalized interventions and/or predictive outcomes, implement the generated personalized interventions and/or predictive outcomes in the digital copy of the subject couple, and/or track the results data of the presented personalized interventions and/or predictive outcomes implemented by the subject. The subject couple may include one of a man and a woman, a woman and a sperm donor, or a man and an egg donor. The system may further include a reproductive family health archive, wherein the reproductive family health archive may include an archive of data collected from each subject couple using the couple-based digital health platform system. The reproductive family health archive may further include data collected from one or more offspring of the subject couple. The system may further include one or more entities in communication with the server via the network, wherein the one or more entities may include healthcare providers, one or more laboratory service providers, one or more pharmacies, and/or one or more IVF providers. The instructions may further include generating alternative personalized interventions and/or predictive outcomes for the subject couple. The multimodal AI and/or ML algorithms may generate the alternative personalized interventions and/or predictive outcomes. The data store may store one or more of data modalities, subject couple health data, other entities data, personalized predictions data, personalized interventions data, and/or reproductive family health archive data.

In another embodiment, a method of using a couple-based digital reproductive health platform is provided. The method may include providing a couple-based digital reproductive health system. The system may include server. The server may include a reproductive health application, ML algorithms, multimodal AI, reproductive health GUI, controller, operating memory, and a communications interface, and wherein the server is accessible via a network. The system may further include a data store in communication with the server. The controller may be configured to execute stored program instructions, which may include collecting data related to a subject couple; analyzing the collected data related to the subject couple; creating a digital copy of the subject couple based on the analyzed collected data; generating personalized interventions and/or predictive outcomes based on the analyzed collected data related to the subject couple; implementing the generated personalized interventions and/or predictive outcomes in the digital copy of the subject couple; presenting personalized interventions and/or predictive outcomes to the subject couple based on results from implementing the generated personalized interventions and/or predictive outcomes in the digital copy of the subject couple; and tracking results data of the presented personalized interventions and/or predictive outcomes implemented by the subject couple. The method may further include collecting data related to a subject couple; analyzing the collected data related to the subject couple; creating a digital copy of the subject couple based on the analyzed collected data; generating personalized interventions and/or predictive outcomes based on the analyzed collected data related to the subject couple; implementing the generated personalized interventions and/or predictive outcomes in the digital copy of the subject couple; presenting personalized interventions and/or predictive outcomes to the subject couple based on results from implementing the generated personalized interventions and/or predictive outcomes in the digital copy of the subject couple; and tracking results data of the presented personalized interventions and/or predictive outcomes implemented by the subject couple.

### BRIEF DESCRIPTION OF THE DRAWINGS

Having thus described the subject matter in general terms, reference will now be made to the accompanying drawings, which are not necessarily drawn to scale, and wherein:

FIG. 1 illustrates a schematic diagram of an example of a reproductive health platform, in accordance with an embodiment of the invention;

FIG. 2 illustrates a schematic diagram of an example of various data modalities of the reproductive health platform shown in FIG. 1, in accordance with an embodiment of the invention;

FIG. 3 illustrates a flow diagram showing an example of how multiple factors may influence fertility outcomes using the reproductive health platform shown in FIG. 1, in accordance with an embodiment of the invention;

FIG. 4 illustrates is a flow diagram showing an example of the application of multimodal AI for the promotion and prevention of reproductive health concerns using the reproductive health platform shown in FIG. 1, in accordance with an embodiment of the invention;

FIG. 5 illustrates a flow diagram showing an example of leveraging exposomic and omics data to develop fertility interventions using the reproductive health platform shown in FIG. 1, in accordance with an embodiment of the invention;

FIG. 6 illustrates a flow diagram showing an example of personalized fertility care through the implementation of digital couples in the reproductive health platform shown in FIG. 1, in accordance with an embodiment of the invention;

FIG. 7 illustrates a block diagram of an example of a reproductive health system for implementing the reproductive health platform shown in FIG. 1, in accordance with an embodiment of the invention;

FIG. 8 illustrates a flow diagram of an example of a method of using the reproductive health platform, which is a couple-based digital reproductive health platform for fertility solutions, in accordance with an embodiment of the invention; and

FIG. 9 shows a screenshot of an example home screen of, for example, a web-based desktop application or a mobile app of the reproductive health platform shown in FIG. 1, in accordance with an embodiment of the invention.

### DETAILED DESCRIPTION

The subject matter now will be described more fully hereinafter with reference to the accompanying drawings, in which some, but not all embodiments of the subject matter are shown. Like numbers refer to like elements throughout. The subject matter may be embodied in many different forms and should not be construed as limited to the embodiments set forth herein; rather, these embodiments are provided so that this disclosure will satisfy applicable legal requirements. Indeed, many modifications and other embodiments of the subject matter set forth herein will come to mind to one skilled in the art to which the subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims.

In some embodiments, the subject matter provides a couple-based digital reproductive health platform and methods.

In some embodiments, the couple-based digital reproductive health platform and methods may be integrated with other corporate partners.

In some embodiments, the couple-based digital reproductive health platform and methods provide a means for using multimodal artificial intelligence (AI) in reproductive health.

In some embodiments, the couple-based digital health platform and methods provide AI combined with multimodal data to address the current challenges in reproductive (including fertility) care with adoption of a prevention framework and holistic couple-based approach over the family health span.

In some embodiments, the couple-based digital reproductive health platform and methods provide data-driven personalized reproductive care by collecting comprehensive multimodal data on reproductive health.

In some embodiments, the couple-based digital health platform and methods may use AI and/or machine learning to collect and analyze both exposome data and biological (or clinical) data about and/or related to the couple.

In some embodiments, the couple-based digital health platform and methods may use AI and/or machine learning to collect and analyze both exposome data and biological (or clinical) data about and/or related to the couple and then build a digital couple that corresponds to a real couple.

In some embodiments, the couple-based digital reproductive health platform and methods may use AI and/or machine learning to make predictions about outcomes that can inform interventions to increase reproductive success.

In some embodiments, the couple-based digital reproductive health platform and methods may provide a reproductive family health archive that may be, for example, a legacy archive of data collected from each couple and potentially across multiple generations for archiving and/or tracing family reproductive and fertility history.

Further, a reproductive health system for implementing the reproductive health platform is provided and wherein the reproductive health system may be provided in a networked computing configuration that includes a couple-based digital reproductive health application and a data store running on an application server and wherein the reproductive health system may be a cloud-based system.

Further, a method of using the reproductive health platform, which is a couple-based digital reproductive health platform for seamless fertility solutions, may be provided.

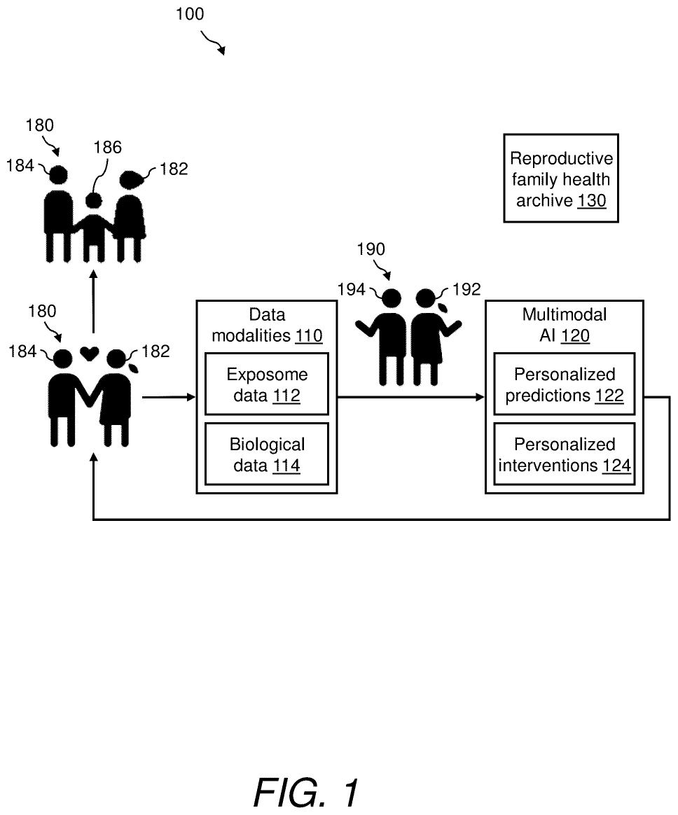

Referring now to FIG. 1 is a schematic diagram of an example of a reproductive health platform 100, in accordance with an embodiment of the invention. Reproductive health platform 100 may be an example of a couple-based digital health platform for providing seamless fertility solutions.

In reproductive health platform 100, multimodal AI may be used to address limitations in the current application of AI in medicine. Reproductive health platform 100 provides a multifaceted framework that uses AI in combination with multimodal comprehensive data sources (i.e., multimodal AI) to address notable shortcomings in reproductive care, for example fertility care. For example, reproductive health platform 100 uses multimodal AI to address the unique challenges and gaps of studying human reproduction and to provide a solution and tool for infertility prevention. Reproductive health platform 100 uses data modalities that can be leveraged and integrated in reproductive care. Reproductive health platform 100 may provide an innovative prevention paradigm that promotes a holistic care model for couples, fosters healthy reproduction across the general population, democratizes fertility care, and reduces costs (derived from reproductive health and medical care) to individuals and society over the family lifespan.

The human reproductive cycle is a highly intricate process composed of multiple complex pathways each requiring molecular, tissue, and organ systems to work synchronously and sequentially in perfect harmony. Human reproduction can be influenced by genetic and environmental factors, both natural and social, and necessitates the cooperation of at least two individuals who contribute gametes. There are several windows of vulnerability throughout the reproductive lifecourse where genes and the environment interact to result in either aberration or adaptation to the development of human life. In addition to the prenatal window, the preconception period is particularly vulnerable to environmental perturbation. Oocytes and sperm carry more than 23 chromosomes—they carry rich epigenetic cargo that programs fertilization, implantation, and successful embryonic growth and development. Environment and lifestyle factors both before and during conception—as well as in pregnancy—program the embryo and fetus and impact offspring health span and life span. The potential for multi- or even trans-generational inheritance of environmental and lifestyle factors has been demonstrated in experimental studies with mounting evidence in humans. In reproductive health platform 100, several gaps in reproductive health may be addressed using a new paradigm with a focus on multimodal AI. In reproductive health platform 100, the proposed paradigm provides for an integrated and holistic framework that considers reproductive health across the lifecourse and through a multigenerational, gene-environment lens.

Referring still to FIG. 1, reproductive health platform 100 may be a couple-based digital reproductive health platform that may be used to assist couples 180 with respect to fertility solutions. Each of the couples 180 may include a woman 182 and a man 184, and with the goal of birthing a baby 186. Reproductive health platform 100 is designed to provide data-driven personalized fertility care by collecting comprehensive multimodal data on reproductive health. For example, information about and/or related to each couple 180 may be applied and/or collected via multiple data modalities 110. Data modalities 110 may include both exposome data 112 and biological data 114, which provide multimodal comprehensive data sources.

Then, based on the information in exposome data 112 and biological data 114 of data modalities 110, a digital (or virtual) couple 190 may be created, which is a digital or virtual representation of a couple 180. That is, digital couple 190 includes a digital woman 192, which is a digital or virtual representation of the woman 182, and a digital man 194, which is a digital or virtual representation of the man 184. In this way, each couple 180 using reproductive health platform 100 has their own corresponding digital couple 190.

Then, using multimodal AI 120, AI may be used in combination with exposome data 112 and biological data 114 of data modalities 110 (i.e., the multimodal comprehensive data sources) and digital couples 190 to model and support the fertility care of couples 180. For example, multimodal AI 120 may be used to process the information in data modalities 110, then apply certain personalized predictions 122 and/or personalized interventions 124 first to the digital couple 190. Then, when positive outcomes are indicated with respect to the digital couple 190, personalized predictions 122 and/or personalized interventions 124 may be presented to the corresponding couple 180 and then acted on by the couple 180. All with the goal of improving the fertility health of the couple 180 and providing the highest possible chance of producing a baby 186.

In other embodiments, a couple 180 may be a same sex couple. In the case of a female couple, in reproductive health platform 100, couple 180 may be a woman 182 (one of the female partners) and a sperm donor. In the case of a male couple, in reproductive health platform 100, a couple 180 may be a man 184 (one of the male partners) and an egg donor.

Further, reproductive health platform 100 may include a reproductive family health archive 130. Reproductive family health archive 130 may be, for example, a legacy archive of data collected from each couple 180 using reproductive health platform 100. Additionally, reproductive family health archive 130 may be an archive of data collected from the offspring of each couple 180 and potentially across multiple generations (e.g., two or three generations). Generally, reproductive family health archive 130 may provide a way by which couples 180 and eventually their children can archive and/or trace their reproductive and fertility history. Including their exposomic, biologic, and medical data, which may be stored, for example, in one secure database or cloud.

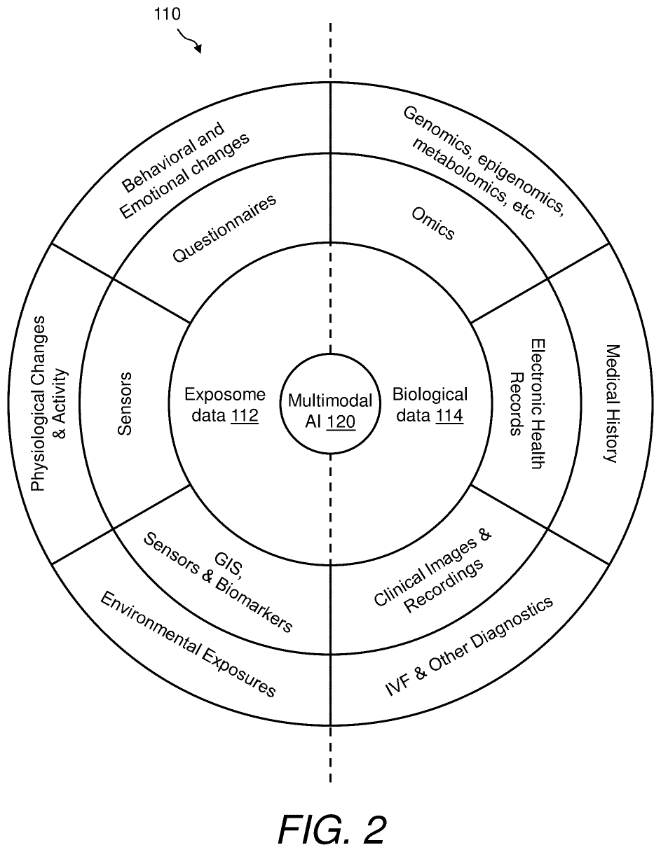

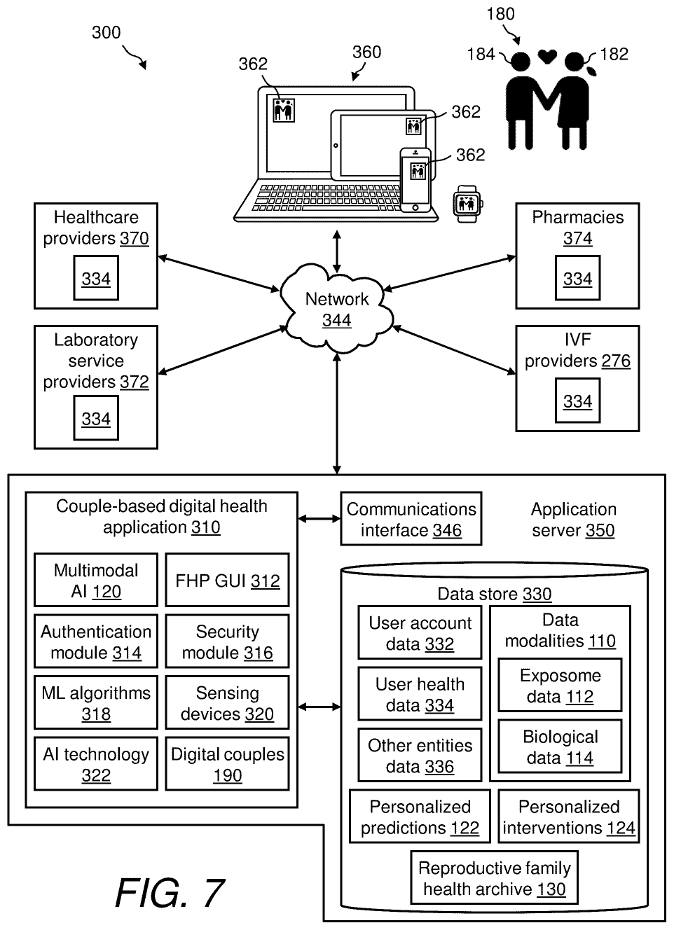

In summary, reproductive health platform 100 may be used to operate in real time with each couple 180 to understand the unique needs of the couple 180 and then provide personalized guidance on lifestyle and behavior factors that are in the couple's control. More details of examples of data modalities 110 are shown in FIG. 2. Further, more details of an example of a networked computing system for implementing reproductive health platform 100 are shown in FIG. 7.

Referring now to FIG. 2 is a schematic diagram of an example of various data modalities 110 of reproductive health platform 100 shown in FIG. 1, in accordance with an embodiment of the invention. Reproductive health platform 100 provides a mechanism for substantially continuous and real time collection of data/information from and/or related to couples 180.

Exposome data 112 of data modalities 110 may include, for example, information collected via questionnaires. For example, questionnaires about behavioral and emotional changes of the couple 180. In another example, exposome data 112 may include information collected via sensors, such as, but not limited to, sensors indicating physiological changes and activity through, for example, a wearable device. In yet another example, exposome data 112 may include information collected via a geographic information system (GIS), electronic sensors (e.g., wearables, such as an iWatch), and/or biomarkers (e.g., temperature, heart rate variability, sleep, steps, direct current potential, oxygen levels, and electroencephalography (EEG), and/or the like). GIS, electronic sensors, and biomarkers may provide, for example, information about environmental exposures. GIS may consist of integrated computer hardware and software that store, manage, analyze, edit, output, and visualize geographic data (geospatial in nature: global positioning system (GPS), remote sensing, etc.)

Electronic sensors used to inform exposome data 112 of data modalities 110 may include, for example, wearable and/or indoor sensors. For example, wearable and/or indoor sensors may be useful devices that objectively collect exposome data 112. Wearable sensors may be used to closely monitor individuals' physical activity and physiological changes including heart rate, body temperature, sleep quality and duration, oxygen levels, and EEG, and/or the like. These sensors may provide more accurate and objective measurements than just questionnaires. Additionally, new phenotyping technology is being developed, such as Beiwe and HOPES, that may make collecting phenotyping data easier through a smartphone-based application. For example, smartphone sensors may be used to collect data associated with variations in mental health (e.g., semantic coherence, behavioral entropy, reaction time). Home sensors in the living spaces of individuals can monitor the indoor and ambient air quality of common pollutants, such as particulate matter 2.5 (PM 2.5), carbon dioxide, radon, and/or volatile organic compounds (VOCs). Surveys have shown that most pregnant women are willing to wear mobile sensors during pregnancy and change behaviors based on personalized digital recommendations. This suggests that tailored advice informed by automated data collection may be an acceptable and effective intervention during conception trying time and pregnancy.

Biological data 114 of data modalities 110 may include, for example, information collected via clinical images and/or recordings information, such as, but not limited to, information from IVF and/or other diagnostics. In another example, biological data 114 may include information collected via electronic health records (EHR), such as medical history information. In yet another example, biological data 114 may include information collected via omics, such as, but not limited to, genomics information, epigenomics information, metabolomics information, and/or the like. Urine, blood, and/or other biological material may be collected to develop a multiomics fertility clock.

Again, exposome data 112 and biological data 114 of data modalities 110 may be collected for both the woman 182 and the man 184 of each couple 180. Further, in the case of a same-sex couple 180 data may be collected on the woman 182 or man 184 and then also on the donor. For example, in the case of a female couple 180, exposome data 112 and biological data 114 of data modalities 110 may be collected for the woman 182 (one of the female partners) and also for the sperm donor. In the case of a male couple 180, exposome data 112 and biological data 114 of data modalities 110 may be collected for the man 184 (one of the male partners) and also for the egg donor.

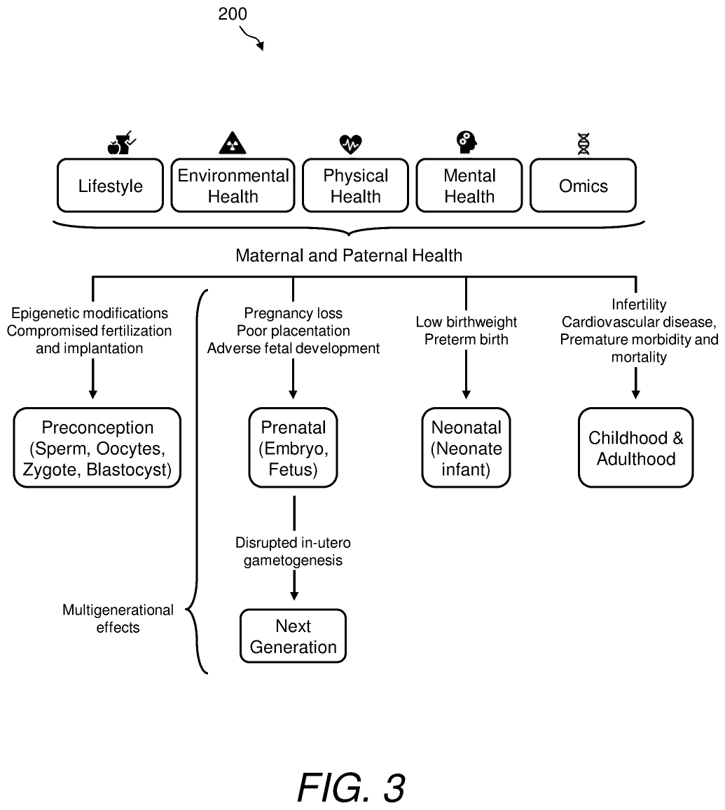

Further to the example, FIG. 3 is a flow diagram 200 showing an example of how multiple factors may influence fertility outcomes using reproductive health platform 100 shown in FIG. 1, in accordance with an embodiment of the invention. That is, FIG. 3 shows the effect of maternal or paternal exposures across the reproductive cycle and generations. Successful pregnancy leading to live birth is the result of countless events happening synergistically, including the right timing of intercourse, good quality oocyte and sperm, a suitable in utero environment for the embryo and fetus to develop, and the female's physiological capacity to carry a fetus to term. The total environment that a couple inhabits, which is comprised of natural (e.g., drinking water, air quality, geographic location, environmental pollutants), behavioral (e.g., diet, lifestyle, product-use patterns), built (e.g., residing buildings, greenspace), social (e.g., mental health, intimate relationship quality, culture, and neighborhood/community-level determinants) elements, plays a critical role in the success of their pregnancy and the subsequent health of their offspring.

In flow diagram 200, lifestyle may be, for example, physical activity, diet, substance use, sleep, and/or the like. Environmental health may be, for example, exposure to chemicals or pollutants in the air, water, food, and/or the like. Physical health may be, for example, acute and chronic health conditions, physiological fitness, and/or the like. Mental health may be, for example, relationships, stress, social environments, traumatic experiences, and/or the like. Omics may be, for example, epigenetics, genetics, proteomics, metabolomics, and/or the like.

The current application of AI in reproductive care mainly focuses on ART procedures that select the best sperm, oocyte, and embryo to transfer back to the female body. while some newer AI algorithms focus on outcome prediction using maternal factors. However, healthy reproduction requires more than the selection of seemingly healthy gametes in the laboratory. For example, subtle epigenetic changes in gametes can have an enormous impact on the success of a pregnancy and the health of the offspring. Growing evidence suggests that lifestyle, nutrition, mental health of both the male and female, and the quality of the couple's relationship all contribute to pregnancy success. Research shows that up to 40% of couples who seek infertility treatment end up conceiving naturally either before treatment begins, in between cycles, or after withdrawing from fertility care. This finding highlights the limitation of only focusing on medical treatment in fertility care and indicates potential vast opportunities for behavioral and psychological interventions to modify and mitigate couples' environmental exposures. Medical treatments are just one example in a broader array of interventions targeting modifiable factors. The narrow focus on a medical model of reproductive disease treatment limits exploration of other possibilities, such as lifestyle interventions, which typically cost less and give the couple the autonomy to control their own health. Moreover, the focus on treatment of disease as opposed to a primary, secondary, and tertiary preventative model has left the reproductive care space limited and controlled by a profit-driven medicalized system that pressures couples to opt into high-technology treatment.

By contrast, and referring now again to FIG. 1, FIG. 2, and FIG. 3, reproductive health platform 100 may be used to provide a more sustainable and person-centered approach than current methods. That is, reproductive health platform 100 may provide an approach that advocates for holistic, personalized reproductive care that optimizes opportunity for natural fertility success while also benefiting the overall health of couples 180 and the future health of their offspring.

For example, reproductive health platform 100 may be used to address the missing link of fathers and fertility as a couple-based concept. In contrast to other medical specialties, such as cardiovascular disease, where the research and/or clinical unit is a single entity (i.e., the patient with disease), human reproduction, by definition, involves at least two individuals, and up to three when considering the offspring. The health of both parents, therefore, influences reproductive success including fertility outcomes and child health. For example, paternal preconception exposure to environmental pollutants is linked to adverse birth outcomes, including preterm birth, low birthweight, and neurodevelopmental outcomes such as autism spectrum disorder. Historically, most attention has been afforded to maternal exposures during pregnancy, with a significant burden of expectations to mitigate risks placed on pregnant women. This is not only contrary to reproductive biological evidence of the role of male's exposures on pregnancy success and offspring health, but has further limited opportunities to include males in the fertility and reproductive process as active partners. Consequently, investigation into paternal health has been largely restricted to occupational exposures and mostly ignored outside of its effects on semen quality. This has contributed to the belief that males are naive contributors of gametes and has resulted in few resources to support or guide them in their reproductive journey.

The more holistic model of reproductive health platform 100 recognizes both maternal and paternal exposures as a complex unit with joint synergistic and potentially antagonistic effects needed to comprehensively address fertility care and reproductive health. Challenges of this holistic approach include the dearth of efforts to translate research evidence into clinical practice, limited data availability in the research field, and lack of useful and user-friendly information for couples. To name a few, many materials designed to inform couples are complex and at a high reading level, resources are primarily directed towards treatment rather than prevention, and there are few-if any-affordable consumer products that minimize harmful exposures and/or focus on prevention. Moreover, information, care provision, and services in the fertility space are highly fragmented and do not address the couple as one unit or facilitate fertility alignment as a partnership.

Accordingly, reproductive health platform 100 may be used to provide solutions to facilitate collection of couple-level data, overcome barriers, improve accessibility, and provide personalized recommendations and treatments based on a prevention framework. This is needed to holistically care for couples' reproductive health across their family-building lifecourse.

Further, reproductive health platform 100 may be used to address the windows of vulnerability for optimal fertility across the lifecourse. There are several critical windows of vulnerability during human reproduction where exposures can exert particularly large impacts on reproductive outcomes compared to other periods. These vulnerable windows include the preconception (for both male and female), prenatal, and postnatal periods, during which environmental exposures can impact pregnancy success and infant-child health (see FIG. 3). For instance, low prenatal folate status in mid-pregnancy have been associated with lower semen quality in male offspring once they reach adulthood. Additionally, there is evidence for multi- and trans-generational effects, wherein exposures incurred by prior generations can influence the health of the index pregnancy and the long-term health span and life span of the offspring. The inability to adequately capture exposures during these different periods and across generations contributes to a critical challenge faced by reproductive care—not being able to identify and quantify risks associated with both male and female modifiable preconception exposures, thereby missing primary, secondary, and tertiary prevention opportunities. Critically, it has further prevented opportunities to identify etiologies of disease in offspring that may have origins in the paternal or maternal preconception period. This data gap has curtailed the understanding of childhood diseases and may be the missing data needed for breakthroughs in the prevention and treatment of pediatric diseases. Accordingly, it may be beneficial to adopt the prevention framework for fertility care provided in reproductive health platform 100.

For example, traditional reproductive care overlooks the importance of infertility prevention and instead focuses on providing high-technology ART when couples present at a fertility clinic for evaluation. By contrast, reproductive health platform 100 facilitates a holistic couple-based prevention framework for fertility care which incorporates multimodal AI models.

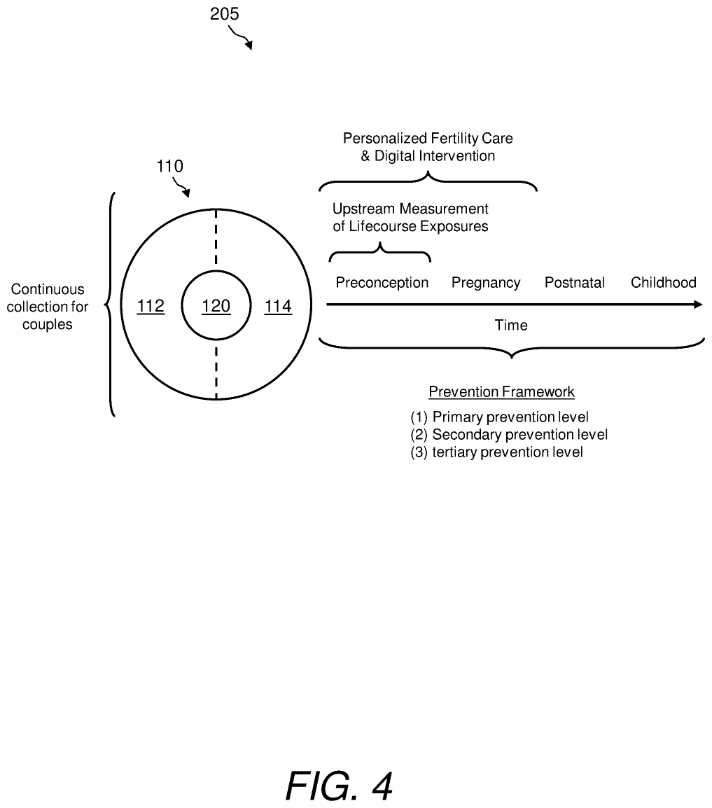

Referring now to FIG. 4 is a flow diagram 205 showing an example of the application of multimodal AI for the promotion and prevention of fertility health concerns across the lifecourse using reproductive health platform 100 shown in FIG. 1, in accordance with an embodiment of the invention.

As with all population-based strategies, this framework may include the following.
(1) a primary prevention level, which may be, for example, adopting and maintaining a healthy fertility lifestyle and reducing exposure to risk factors;
(2) a secondary prevention level, which may be, for example, early identification of couples as risk and interventions to reduce exposure to modifiable factors; and
(3) a tertiary prevention level, which may be, for example, treatment-related burden among couples requiring infertility services.

Further, reproductive health platform 100 may include a digital therapeutic tool (also known as a Software as a Medical Device (SaMD)) that may be used to collect multimodal data (e.g., data modalities 110) and apply machine learning algorithms to tailor and personalize interventions (e.g., personalized interventions 124) to couples 180. Potentially improving the reach and scale of a prevention framework while also targeting populations at risk. In this context, the primary prevention of reproductive health platform 100 focuses on adopting strategies to reduce exposure to fertility-related risk factors in the general population and maintaining a healthy fertility lifestyle to decrease the risk of potentially adverse reproductive outcomes. Altering dietary behaviors to limit processed foods and choosing higher quality items, increasing physical activity, improving sleep quality and mental health, among other lifestyle changes, can be considered examples of primary prevention. Secondary prevention involves targeting individuals and couples with high risk factors for adverse reproductive outcomes, such as infertility. The goal is to identify high-risk individuals earlier to formally intervene to reduce exposure to those risk factors in both members of the couple 180 through personalized interventions 124. Lastly, tertiary prevention aims to reduce treatment-related burdens among couples while receiving fertility services. The focus of tertiary prevention is to reduce the risks of harm due to fertility treatment and improve outcomes while minimizing the long-term health sequalae to couples and their offspring.

Again, reproductive health platform 100 may provide a digital therapeutic tool to facilitate a holistic, couple-based prevention platform that applies multimodal AI 120 to reduce risk, improve outcomes, prevent disease, identify more high-risk individuals, and benefit a greater range of vulnerable populations who otherwise lack access to treatment.

Referring still to flow diagram 205 of FIG. 4, with respect to data modalities 110, reproductive health platform 100 may be used to harmonize and synthesize multimodal data, and thereby make it possible to shift the current fertility paradigm of high-technology disease treatment to that of an AI-driven, couple-based holistic care that emphasizes prevention and early intervention. For example, prevention care may incorporate data from multiple comprehensive data sources (e.g., exposome data 112 and biological data 114 of data modalities 110) and apply the resulting deep phenotyping to predict reproductive outcomes (e.g., personalized predictions 122) and inform personalized interventions 124 to couples 180 as a unit. In reproductive health platform 100, AI technologies, such as wearable sensors and at-home monitors, may be used for data collection by continuously monitoring physiological and environmental changes in an individual's ambient and built environment. Linking longitudinal data from multiple sources with repeated or continuous data collection on a large scale across the population is the foundation of holistic, preventative reproductive care that can be integrated across the lifecourse of the couple and their future family. Below we discuss several data modalities for consideration in such a multimodal AI-driven prevention framework of fertility care.

For example, with respect to data modalities 110 of reproductive health platform 100, exposome data 112 is considered the totality of environmental (non-genetic) exposures over an individual's lifecourse. Exposome data 112 may include a breadth of exposures that can be generally divided into three domains: internal, specific external, and general external. The internal factors are at the individual level, such as physiology, age, and mental health; specific external factors include lifestyle, diet, physical activity, and occupational and environmental pollutant exposures; general external factors are social predictors such as race, education, and household income. Some exposome data 112 may be obtained through self-reported questionnaires and questionnaire-based professional scales (e.g., mental health scales) which are not usually collected in clinical settings. Environmental pollutant exposures can be directly measured as biomarkers or estimated via self-reported occupation, residential location determined by GIS, and passive monitoring by wearable and indoor sensors. Exposome data 112 of the couple 180 plays an important role in achieving successful pregnancy and offspring health, particularly during sensitive windows when exposures have greater impacts. However, it is challenging to collect exposome data 112 in traditional epidemiologic research settings due to loss to follow-up and high costs. Accordingly, reproductive health platform 100 provides a digital platform that directly interacts with couples 180 prior to pregnancy. Reproductive health platform 100 also allows for cost-effective, active data collection and has the capacity to collect, link, and store a wide range of exposome data 112 for current and future use (again see FIG. 4).

Currently, clinical data (or biological data) in reproductive care is derived from multiple fragmented sources. For example, in the fertility clinic setting, EHR data covering medical history is sex-specific and stores data from females and males separately instead of as a couple. Additionally, results of lab diagnostics from external laboratories exist independently from EHR and are not always imported, harmonized, or analyzed to understand the complexity of care. Similarly, in IVF settings, the number and quality of the obtained gametes, fertilized eggs, and embryos for implantation are stored independently in clinic databases, disconnected from other data, including obstetric and birth records, and neonatal and pediatric health outcomes data.

By contrast, reproductive health platform 100 may be used to link clinical data in one universal platform at the couple level rather than the individual level and thereby provides a comprehensive history of couples' medical care and conditions over their lifecourse in the form of biological data 114 (or clinical data) of data modalities 110. Accordingly, reproductive health platform 100 may be used to facilitate the selection of personalized interventions 124 to improve their reproductive success (again see FIG. 4).

Other medical fields have had success in combining multiple sources of clinical data. For example, in cardiovascular medicine, combining EHR data with imaging to identify pulmonary embolism has been shown to outperform techniques using individual data modalities alone. In like manner, reproductive health platform 100 may be used to harmonize, synthesize, and analyze reproductive health data, irrespective of clinic choice, procedures, or outcomes, that yields an archive of personal level health information on family building within couples across time. In reproductive health platform 100, this reproductive health archive may be owned by the couple 180. This may allow them to access personalized insights that they could track and share with their care providers over the lifecourse for them and their offspring.

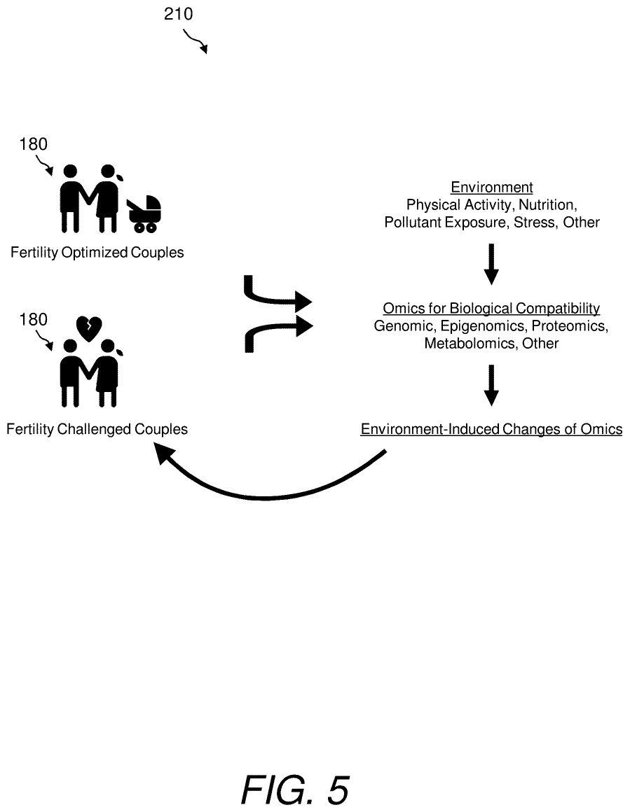

Referring now to FIG. 5 is a flow diagram 210 showing an example of leveraging exposomic and omics data to develop fertility interventions using reproductive health platform 100 shown in FIG. 1, in accordance with an embodiment of the invention. For example, modifying omics to improve biological compatibility of couples 180.

Omics is one example of biological data 114 (or clinical data) of data modalities 110. Omics can be defined as large-scale, data-rich biological measurements of the genome and its expression. Omics data captures an individual's fundamental biological expression, including their genome, epigenome, proteome, microbiome, and metabolome, among others. Omics data benefits and improves the understanding of human health at microscopic levels. In reproductive health platform 100 omics data allows for the development of precise and individualized treatment strategies. In fertility clinic settings, preimplantation genetic screening and testing have been used to select embryos that have no chromosomal abnormalities and lack specific genes that are related to inheritable diseases. Additionally, genetic assessment is often used to screen for genetic drivers of infertility. Although epigenetics is often neglected in current reproductive care practice, mounting evidence has shown that epigenetics (including DNA methylation, histone modification, and non-coding RNAs) and proteomics play a critical role in infertility and the multi- and trans-generational effects of environmental exposure. By contrast, in reproductive health platform 100 epigenetics may be included as a factor with respect to reproductive care for couples 180.

Further, metabolomics is another example of biological data 114 (or clinical data) of data modalities 110 of reproductive health platform 100. Metabolomics may be used to help predict fertility treatment outcomes. Deep phenotyping on multiple omics (i.e., multiomics) may be used to provide insights into an individual's- and couple's-biological uniqueness permitting the development of personalized prevention at the primary, secondary, and tertiary levels.

Omics data have been applied to perinatal research to predict outcomes. For example, omics as biological indicators of an individual's health may be used to predict fertility outcomes. The potential for intervening omics, particularly the modifiable proteomics and metabolomics, to improve fertility care has yet to be fully comprehended. From a reproductive evolutionary biology perspective, biological compatibility of couples might determine overall fit in the reproduction of healthy offspring. There has been some discussion on the genetic determination of partner selection and offspring health; however, most of the evidence is outdated and not validated in large human populations. By contrast, in reproductive health platform 100, the reproductive success of couples 180 may depend on their biological compatibility, not only genetics (which is largely non-modifiable) but also potential modifiable factors, such as epigenetics, proteomics, and metabolomics (again see FIG. 5).

In reproductive health platform 100, multiomics profiling may be used as a potential tool for investigating biological compatibility of couples by obtaining and comparing omics data between couples with and without reproductive success or within the same couple across time with repeat measurements. Further, multiomics data modelling may be used to learn how to improve biological compatibility of couples or even “engineer” biological compatibility via synthetic derivatives of proteomic or metabolomic expression (again see FIG. 5). In reproductive health platform 100, the multiomics model of reproductive-biological compatibility of couples is novel and has the potential to improve fertility success by improving upstream biological inputs and/or modify risk factors for infertility and facilitate risk population management. Taking this a step further, measuring changes in proteomics or metabolomics across time following changes in exposures, such as nutrition, exercise, and pollutants, may provide clues on how modifying the environment (i.e., “engineering” the environment) may alter couples' biological compatibility (i.e., couple-based multiomics profile). Ultimately, it may be possible to intervene on these corresponding multiomics directly to improve couples' fertility outcomes (again see FIG. 5).

Reproductive health platform 100 may provide a means for implementing multimodal AI in reproductive health. For example, multimodal data may be used to capture the totality of a couple's history (genetic and environmental) and real-time risk factor profile. Incorporating multimodal data (e.g., data modalities 110) into AI algorithms (e.g., multimodal AI 120) provide novel opportunities to inform couple-based personalized interventions 124 and treatments. Using reproductive health platform 100, below describes several opportunities for the future of multimodal AI in reproductive care focused on a holistic couple-based prevention paradigm for fertility health across the lifecourse.

For example, upstream data curation may be implemented in reproductive health platform 100. One challenge in traditional reproductive care is that data in clinical settings is primarily downstream. Upstream data (e.g., exposure during the preconception period) is harder to obtain, and current methods often rely on potentially biased retrospective recall. Moreso, individuals only seek medical care if they have difficulty conceiving a pregnancy, experience a pregnancy loss, or after finding out they are pregnant. Consequently, clinicians may treat and manage downstream events only, while upstream factors that are critical drivers of disease and adverse outcomes remain out of reach. AI provides unique opportunities to capture upstream data through the development of AI platforms that monitor couples' health before pregnancy (see FIG. 4). Leveraging digital applications and wearable devices to track couples' physiological changes and behaviors in the preconception period allows for the collection of upstream data on crucial predictors for various fertility outcomes across the continuum of the reproductive life cycle. Doing so optimizes opportunities for primary, secondary, and tertiary prevention of fertility problems, as described in FIG. 4.

Further, reproductive health platform 100 may provide a means for hidden data monitoring. Many variables influencing human reproduction are difficult to uncover, such as the timing of ovulation. The subtle, hard-to-capture physiological changes that are characteristic of ovulation and the large between- and within-person variations in timing complicate prediction for the fertile window outside of clinical settings where females are actively undergoing fertility treatment. The use of wearable devices to closely monitor physiological changes (e.g., basal temperature changes and hormonal changes of the individuals) combined with AI algorithms (e.g., multimodal AI 120) may be used to address and improve this fundamental challenge in reproductive care. Moreover, AI may also provide opportunities to capture behavioral changes (e.g., intercourse frequency and/or adherence to lifestyle interventions) that are valuable predictors of pregnancy probability and can inform couples' decision to use contraception. Previous studies have attempted to use wearable devices to predict fertile windows. Additionally, multimodal AI may be used to help identify vulnerable populations to increase the resources allocated to them and address health disparities.

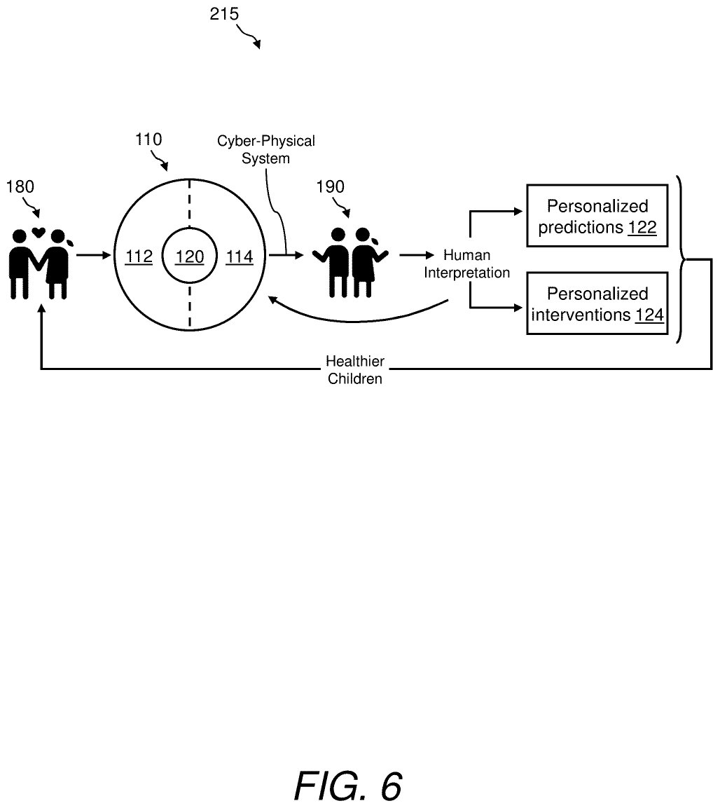

Referring now to FIG. 6 is a flow diagram 215 showing an example of personalized fertility care through the implementation of digital couples 190 in reproductive health platform 100 shown in FIG. 1, in accordance with an embodiment of the invention.

For example, reproductive health platform 100 may provide for personalized fertility care for couples. For example, incorporating data from multiple sources (e.g., data modalities 110), including exposome data 112, biological (or clinical) data 114, electronic medical records, omics, and sensors, provide insight on the unique health status that each couple 180 may face at the start of their fertility journey. Capturing complex, holistic, multimodal data and applying AI algorithms (e.g., multimodal AI 120) to inform the health of the couple no matter where they find themselves on the fertility journey allows for optimal personalized preventative as well as precision medicine treatment (i.e., pharmaceuticals and medical interventions) opportunities (see FIG. 6).

Combining omics with biological (or clinical) data 114 allows for the consideration of a couples' biological features and medical conditions, while data capturing the exposome data 112 from external devices can identify potential environmental factors influencing couple health. Given that each couple 180 has a unique combination of exposures and omics, this powerful approach has the potential to provide insights that simply cannot be captured by population-level data. Multimodal data (e.g., data modalities 110) from couples 180 may reveal concerns specific to the individuals experiencing them and allows for more targeted and personalized holistic care for couples as they navigate their reproductive journey. Furthermore, by collecting multimodal data from the couples 180 over time, reproductive health platform 100 may be used to gain valuable insight into responses to treatment that can guide subsequent care (again see FIG. 6).

Further, reproductive health platform 100 may provide a means for digital health interventions for optimal reproductive health among couples 180. Reproductive health platform 100 provides a software platform that collects, harmonizes, and analyzes data on the medical and reproductive histories of couples 180. The total environment of couples 180 may be harnessed to inform personalized digital interventions (e.g., personalized interventions 124) to help couples 180 attain fertility health and achieve successful reproduction (again see FIG. 6). Compared to medical interventions, digital interventions are cost-effective, more efficient, easy to integrate, and can incorporate deep and comprehensive data that is scalable to millions.

Digital therapeutics (DTx) are recognized as medical devices (prescription) by U.S. Food and Drug Administration (FDA). Several DTx are applied in other medical settings for disease treatment and management. Further, smartphone-driven digital interventions have been applied to chronic disease management and may be useful in reproductive health platform 100.

The potential of DTx to augment population-level fertility health through widespread accessibility and large scalability has not yet been realized. Smartphone-based digital interventions are convenient, accessible, and cost-effective. Digital interventions that target lifestyle changes are cheaper compared to clinical treatments in reproductive care, especially IVF. Thus, clinicians may consider using digital interventions as a primary care option when treating couples that are trying to conceive, subfertile, or infertile, with the recommendation to allow couples time to adjust their behaviors to facilitate natural conception. Clinicians may also choose to provide digital interventions while a couple undergoes fertility treatment in an attempt to increase their potential to conceive and have a successful pregnancy. Further, because reproductive health platform 100, being a digital platform, may be used to track progress in real-time, reproductive health platform 100 may serve to motivate couples 180 to comply with interventions to improve outcomes. This may be done by visually demonstrating the consequences of limited compliance to the intervention versus their predicted probability of conception with full adherence.

Further, reproductive health platform 100, being a digital platform, may offer opportunities to consult with and find support in other couples who have struggled with fertility concerns, allowing the couple to learn from the strategies of previous successful cases, share their own feelings with couples who are on the same path of reproductive care, and establish a support network. Virtual group coaching has shown success in other areas of healthcare, including in motivation and maintenance of behavior change related to weight loss.

“Digital twins” were first used in industry at the beginning of the 21st century to optimize product lifecycle management. In reproductive health platform 100, the goal is to build a digital copy or twin (digital woman 192 or digital man 194) of a real individual (woman 182 or man 184) by integrating data from multiple sources (e.g., data modalities 110) in real time to assist decision-making and predictive evaluations for the physical patient. Digital twins have only recently entered the medical field. The core concepts of a digital twin rely on several major concepts in computer sciences, including AI, the Internet of Things (IoT), and closed-loop optimization (CLO). AI emulates human reasoning using big data processing and pattern recognition. IoT facilitates the creation of a digital profile under real-world scenarios and performs rapid data synchronization between physical and digital twins. Combining AI with IoT to create the cyber-physical system (CPS) (again see FIG. 6), which establishes bidirectional data exchange between the digital and physical twins on a continuous or periodic basis. Adding CLO allows the virtual twin to quickly reconfigure and adopt the properties of its physical twin. The digital twin can then be used to identify modifiable risk factors for early identification or detection, evaluate, diagnose, and predict disease while testing and optimizing potential solutions before the initiation of an intervention.

In reproductive health platform 100, digital twins (e.g., digital woman 192 or digital man 194 of digital couple 190) may be applied to monitor the status of the real couple 180 as a unit and collect and fuse male and female data from multiple sources in real time. The digital twin can then be used to model and predict the couple 180's probability of pregnancy or treatment success and suggest which interventions (or environmental “engineering”) have the best probability of success (again see FIG. 6).

In operation, and referring now again to FIG. 1 through FIG. 6, reproductive health platform 100 receives data from multiple sources, incorporates data about the couple 180, analyzes the data using machine learning algorithms (e.g., multimodal AI 120), and makes predictions (e.g., personalized predictions 122) about outcomes that can inform interventions (i.e., personalized interventions 124—lifestyle changes of the female, male, or both) to increase reproductive success. Moreover, multimodal data capture, curation, and storage serves as an evidence database from which to inform future AI models. Due to the continuous nature of data collection, reproductive health platform 100 may then be used to track behavioral changes or the effectiveness of an intervention over a specified duration of follow-up.

This data on post-intervention behaviors and exposures may serve as a feedback mechanism, informing the modification of personalized interventions 124 to optimize their efficacy, improve health outcomes, and inform the development of future interventions. For example, if man 184 of a couple 180 smokes and woman 182 of the couple 180 is physically inactive, a personalized intervention 124 may include smoking cessation for man 184 and moderate exercise with coaching for woman 182 via reproductive health platform 100. However, if, during the follow-up period, the couple 180 does not adhere to the personalized intervention 124, multimodal AI 120 may be used to suggest alternative personalized interventions 124 targeting lifestyle and other environmental factors (e.g., changing to a healthier, but realistic diet) to improve their chances of pregnancy. This personalized, couple-based approach of reproductive health platform 100 facilitates optimization of the reproductive outcome of couples 180.

In reproductive health platform 100, examples of personalized predictions 122 of multimodal AI 120 may include, but are not limited to, the following: ovulatory potential (woman 182), sperm parameters/health (man 184), and couple 180's overall capacity for reproductive success using fertility. For example, use multimodal AI 120 to analyze the data modalities 110 of the couple 180 to determine the woman 182's opportunity for success. Reproductive health platform 100, may use predictive scores from the multimodal data collection to determine the woman 182's physiologic, psychologic, biologic, and environmental capacity for reproductive success. personalized predictions 122 may include, but are not limited to, physiologic state (e.g., heart rate variability) sleep, stress, exercise, nutrition/diet, chemicals, exposures in the environment, sexual frequency and sexual health, reproductive symptoms, and the like.

Further, examples of personalized interventions 124 of multimodal AI 120 may include, but are not limited to, the following: stress management, meditation, increasing steps, changing and swapping out products, supplements, communication with partner, intimacy and sexual health improvements, reduction in blue light/night light, increase in sunlight/daylight, air purification, water purification, dietary management, exercise and endurance workouts, music and art exposure, blue space and green space exposure, and the like.

Referring now to FIG. 7 is a block diagram of an example reproductive health system 300 that may be used to implement reproductive health platform 100 shown in FIG. 1, in accordance with an embodiment of the invention. In this example, reproductive health system 300 may be provided in a networked computing configuration that includes a couple-based digital health application 310 and a data store 330 running on an application server 350. Further, reproductive health system 300 may be a cloud-based system. In one example, reproductive health system 300 and/or reproductive health platform 100 may operate using a subscription model.

At application server 350, couple-based digital health application 310 may further include multimodal AI 120, a fertility health platform (FHP) graphical user interface (GUI) 312, an authentication module 314, a security module 316, ML algorithms 318, sensing devices 320, AI technology 322, and digital couples 190. Further, user account data 332, user health data 334, other entities data 336, data modalities 110 including both exposome data 112 and biological data 114, personalized predictions 122, personalized interventions 124, and reproductive family health archive 130 may be stored at data store 330. Additionally, application server 350 may be accessible via a network 344. Network 344 may be, for example, a local area network (LAN), a wide area network (WAN), and/or a cellular network for connecting to the Internet or to an Intranet. Application server 350 may connect to network 344 by any wired and/or wireless means. Application server 350 may include, for example, a communications interface 346.

ML algorithms 318 may be used to analyze the data (data modalities 110 including both exposome data 112 and biological data 114) of each of the couples 180. Sensing devices 320 may be, for example, wearable sensors that may provide physiological data, such as wrist skin temperature, heart rate, heart rate variability, respiratory weight, and skin perfusion. AI technology 322 may be, for example, sensing devices 320 (e.g., wearable sensors) and at-home monitors.

A plurality of couples 180 may be associated with the reproductive health system 300 and wherein each couple 180 may include a woman 182 and a man 184. In reproductive health system 300, couples 180 may be any women 182 and/or men 184 seeking help in the area of fertility and/or reproduction health. The couples 180 may access couple-based digital health application 310 at application server 350 via their respective user computers 360 and network 344. User computers 360 may be any computing device, such as, but not limited to, a desktop computer, a laptop computer, a handheld computing device, a mobile phone (or smart phone), a tablet device, a smartwatch, and the like. Any information about couples 180 may be stored in user account data 332 and/or user health data 334 at data store 330 of application server 350. For example, user account data 332 may include a user profile for each woman 182 and man 184 of each couple 180. Each user profile may include, for example, account information, username, user/couple name, user/couple credentials, user payment information, and the like.

In one example, couples 180 may interact with the couple-based digital health application 310 using FHP GUI 312 at application server 350. In this example, FHP GUI 312 may be a web-based GUI that is accessible via network 344. For example, couple-based digital health application 310 at application server 350 may be a software application that may be implemented as a web application and run in a web browser, such as Google Chrome or Microsoft Edge.

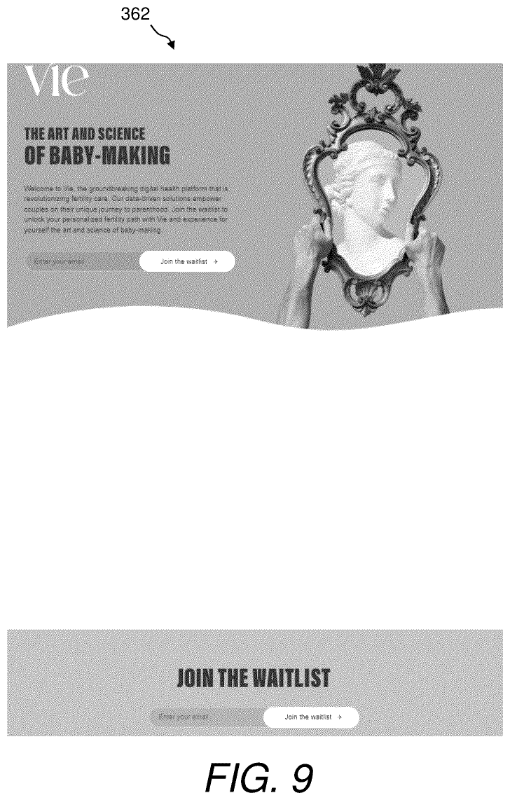

A FHP desktop application 362 or FHP mobile app 362 may be installed and running on each of the user computers 360. A screenshot of an example of the home screen of, for example, a web-based FHP desktop application 362, or FHP mobile app 362, is shown in FIG. 9.

FHP desktop application 362 and FHP mobile app 362 is hereafter called FHP mobile app 362. In this example, FHP mobile app 362 may be implemented, for example, as a .NET application, a desktop application, a mobile app, an application program interface (API), and the like. When configured as a mobile app, FHP mobile app 362 may be designed to operate on any device platform, including for example, Windows, Android, Apple, and the like. Accordingly, couples 180 may interact with the couple-based digital health application 310 using FHP mobile app 362 at their user computer 360 (e.g., smart phone or tablet device).

Referring still to FIG. 7, reproductive health system 300 may also include one or more healthcare providers 370, one or more laboratory service providers 372, one or more pharmacies 374, and one or more IVF providers 376. Further, the healthcare providers 370, laboratory service providers 372, pharmacies 374, and IVF providers 376 may access couple-based digital health application 310 via, for example, the FHP mobile app 362.

Healthcare providers 370 may be any healthcare providers with expertise in fertility and/or reproduction health. Healthcare providers 370 may be assessable in person or via telehealth. Using FHP mobile app 362, healthcare providers 370 may have access to user health data 334 of couples 180. Laboratory service providers 372 may be any entities providing lab services (e.g., lab tests performed on specimens) that may be used to support healthcare services. Pharmacies 374 may be any entities where medicinal drugs are dispensed and sold. Examples of pharmacies may include, but are not limited, Walgreens (Deerfield, IL), CVS (Woonsocket, RI), and Right Aid (Camp Hill, PA). IVF providers 376 may be any entities that provide IVF services.

Authentication module 314 of couple-based digital health application 310 may be used to manage the authentication process of any entities of reproductive health system 300, such as couples 180, healthcare providers 370, laboratory service providers 372, pharmacies 374, and IVF providers 376. For example, when the couple 180 signs into the couple-based digital health application 310, a standard authentication process may be performed that allows the couple 180 to access couple-based digital health application 310. User sign-in may occur any number of ways. In one example, the couple 180 may use a web browser to access FHP GUI 312 of couple-based digital health application 310 and enter credentials (e.g., username and password).

In another example, the couple 180 may use FHP desktop application 362 or FHP mobile app 362 to enter his/her credentials. In yet another example, the user sign-in process may occur automatically when the couple 180 starts FHP mobile app 362. As couples 180 are authorized to access reproductive health system 300, user information may be stored in user account data 332 in data store 330. Similarly, as other entities (e.g., healthcare providers 370, laboratory service providers 372, pharmacies 374, and IVF providers 376) access reproductive health system 300, information may be stored in other entities data 336 in data store 330.

Security module 316 of couple-based digital health application 310 may be used to perform any system security functions with respect to keeping secure the contents of data store 330 and/or any other information with respect to reproductive health system 300. Security module 316 may use standard security techniques, such as encryption, secure hashtags (or hash tags), and the like. Data store 330 may be, for example, data repositories (like databases) and/or flat files that can store data. Further, reproductive health system 300 is not limited to one data store 330 only. Reproductive health system 300 may include multiple data stores 330. Further, data store 330 may be provided on a data server that is separate from application server 350.

Further, certain privacy requirements may exist in reproductive health system 300 that supports reproductive health platform 100. For example, multimodal data may require collecting and harmonizing data from complex sources, including fertility clinics, laboratories, digital platforms that assess lifestyle and environments, wearable devices, and sensor data. Combining numerous complex sources pose immense challenges in data privacy compared to data from a single clinical source. Research has shown that using data from small regions and populations only allows for the re-identification of participants in large datasets. Accordingly, security module 316 may be designed to ensure privacy in multimodal models, including differential privacy, federated learning, homomorphic encryption, swarm learning, and edge computing, all of which can be used complementarily. Further, it is beneficial to incentivize data sharing across sectors while preserving patient privacy.

Communications interface 346 at application server 350 may be any wired and/or wireless communication interface for connecting to a network (e.g., network 344) and by which information may be exchanged with other devices connected to the network. Examples of wired communication interfaces may include, but are not limited to, USB ports, RS232 connectors, RJ45 connectors, Ethernet, and any combinations thereof. Examples of wireless communication interfaces may include, but are not limited to, an Intranet connection, Internet, ISM, Bluetooth® technology, Bluetooth® Low Energy (BLE) technology, Wi-Fi, Wi-Max, IEEE 402.11 technology, ZigBee technology, Z-Wave technology, 6LoWPAN technology (i.e., IPv6 over Low Power Wireless Area Network (6LoWPAN)), ANT or ANT+ (Advanced Network Tools) technology, radio frequency (RF), Infrared Data Association (IrDA) compatible protocols, Local Area Networks (LAN), Wide Area Networks (WAN), Shared Wireless Access Protocol (SWAP), any combinations thereof, and other types of wireless networking protocols.

Referring still to FIG. 7, reproductive health system 300 may operate in a client/server computing architecture, which is well known. In this example, couple-based digital health application 310 at the application server 350 may be the server component of reproductive health system 300, while FHP mobile app 362 at each of the user computers 360 may be the client component of reproductive health system 300. In other words, FHP mobile app 362 at each of the user computers 360 is the counterpart to couple-based digital health application 310 at application server 350.

Additionally, application server 350 may be any networked computing configuration as long as it is accessible via network 344 by other entities of reproductive health system 300, such as couples 180, healthcare providers 370, laboratory service providers 372, pharmacies 374, and IVF providers 376. For example, reproductive health system 300, and more particularly the couple-based digital health application 310 on application server 350, may support a cloud computing environment. In a cloud computing environment, application server 350 may be the cloud server. Further, couple-based digital health application 310 is not limited to running on one application server 350 only. Reproductive health system 300 may include multiple application servers 350 (or cloud servers) to ensure high availability of computing resources.

Generally, and referring still to FIG. 7, couple-based digital health application 310 may be a software application that provides a means of using multimodal AI 120 and FHP mobile app 362 to manage healthcare providers 370, laboratory service providers 372, pharmacies 374, and IVF providers 376 to assist couples 180 with respect to their fertility and/or reproduction health.

Additionally, using couple-based digital health application 310 and FHP mobile app 362, ongoing communication between entities of reproductive health system 300 may occur. For example, using FHP mobile app 362, push notifications may be automatically generated to couples 180 and confirmation notifications may be returned from couples 180 to couple-based digital health application 310.

Multimodal AI 120 may be used to process all the information generated and/or received by couple-based digital health application 310 and thereby manage the overall operations of reproductive health system 300.

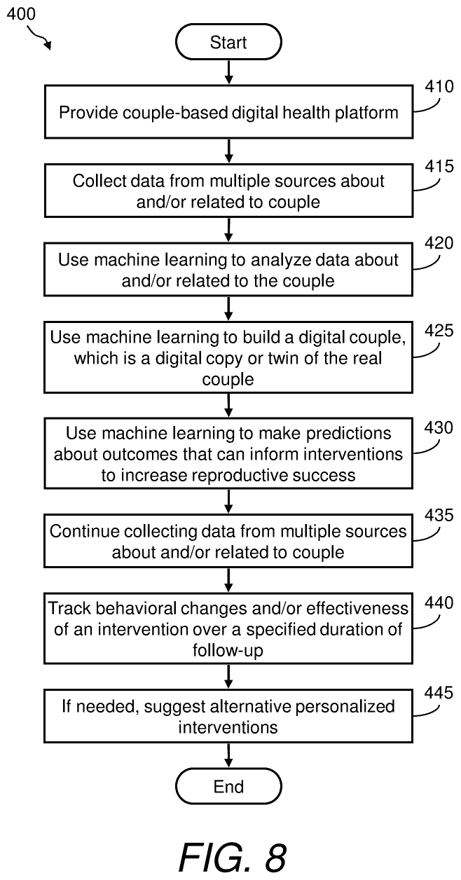

FIG. 8 illustrates a flow diagram of an example of a method 400 of using reproductive health platform 100, which is a couple-based digital reproductive health platform for seamless fertility solutions, in accordance with an embodiment of the invention. Method 400 may include, but is not limited to, the following steps and in any order.

At a step 410, a couple-based digital health platform is provided. For example, the reproductive health platform 100 described above in FIG. 1 through FIG. 7 may be provided. Further to the example, reproductive health system 300 shown in FIG. 7 that supports reproductive health platform 100 may be provided.

At a step 415, data from multiple sources is collected about and/or related to the couple. For example and referring now to FIG. 2, both exposome data 112 and biological data 114 of data modalities 110 may be collected, which provide multimodal comprehensive data sources about and/or related to couple 180.

At a step 420, machine learning is used to analyze data about and/or related to the couple. For example, multimodal AI 120 and/or ML algorithms 318 may be used to analyze both exposome data 112 and biological data 114 of data modalities 110, which is data about and/or related to the couple 180.

At a step 425, machine learning is used to build a digital couple, which is a digital copy or twin of the real couple. For example, multimodal AI 120 and/or ML algorithms 318 may be used to build a digital couple 190, which is a digital copy or twin of the real couple 180. That is, ML algorithm 318 may be used to build a digital copy or twin (digital woman 192 or digital man 194 of digital couple 190) of a real individual (woman 182 or man 184 of couple 180) by integrating data from multiple sources (e.g., data modalities 110) in real time to assist decision-making and predictive evaluations for the physical patient.

At a step 430, machine learning is used to make predictions about outcomes that can inform interventions to increase reproductive success. For example, multimodal AI 120 and/or ML algorithms 318 may be used to makes predictions (e.g., personalized predictions 122) about outcomes that can inform interventions (e.g., personalized interventions 124, such as lifestyle changes of the female, male, or both) to increase reproductive success. In one example, if man 184 of a couple 180 smokes and woman 182 of the couple 180 is physically inactive, a personalized intervention 124 may include smoking cessation for man 184 and moderate exercise with coaching for woman 182 via reproductive health platform 100.

At a step 435, continue to collect data from multiple sources about and/or related to the couple. For example and referring now to FIG. 2, continue to collect both exposome data 112 and biological data 114 of data modalities 110, which provide multimodal comprehensive data sources about and/or related to couple 180. Moreover, multimodal data capture, curation, and storage serves as an evidence database from which to inform future AI models.

At a step 440, behavioral changes and/or the effectiveness of an intervention is tracked over a specified duration of follow-up. For example, multimodal AI 120 and/or

ML algorithms 318 may be used to track behavioral changes or the effectiveness of an intervention over a specified duration of follow-up.

At a step 445, if needed, alternative personalized interventions are suggested. For example, if man 184 of a couple 180 smokes and woman 182 of the couple 180 is physically inactive, a personalized intervention 124 may include smoking cessation for man 184 and moderate exercise with coaching for woman 182 via reproductive health platform 100. However, if, during the follow-up period, the couple 180 does not adhere to the personalized intervention 124, multimodal AI 120 may be used to suggest alternative personalized interventions 124 targeting lifestyle and other environmental factors (e.g., changing to a healthier, but realistic diet) to improve their chances of pregnancy.

This personalized, couple-based approach of reproductive health platform 100, reproductive health system 300, and method 400 facilitates optimization of the reproductive outcome of couples 180.

Referring now to FIG. 9 is a screenshot of an example of the home screen of, for example, a web-based FHP desktop application 362 or FHP mobile app 362.

In summary and referring now again to FIG. 1 through FIG. 9, reproductive health platform 100, reproductive health system 300, and method 400 provide AI combined with multimodal data to address the current challenges in fertility care with adoption of a prevention framework and holistic couple-based approach over the family health span. For example, personalized interventions 124 (i.e., digital interventions) powered by multimodal AI 120 for couples 180 has the potential to achieve equity in reproductive care. A shift in the current treatment-centered medical paradigm is necessary to embrace a future of couple-focused fertility health across the reproductive lifespan driven by multimodal AI innovations. The unique data capture of reproductive health platform 100, through a combination of omics and exposomics collection, results in a biological fingerprint which represents the fertility profile and fertility health and potential of each individual of couple 180.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

The terms “comprise,” “comprises,” “comprising,” “include,” “includes,” and “including,” are intended to be non-limiting, such that recitation of items in a list is not to the exclusion of other like items that may be substituted or added to the listed items.

Terms like “preferably,” “commonly,” and “typically” are not utilized herein to limit the scope of the claimed embodiments or to imply that certain features are critical or essential to the structure or function of the claimed embodiments. These terms are intended to highlight alternative or additional features that may or may not be utilized in a particular embodiment of the present invention.

The term “substantially” is utilized herein to represent the inherent degree of uncertainty that may be attributed to any quantitative comparison, value, measurement, or other representation and to represent the degree by which a quantitative representation may vary from a stated reference without resulting in a change in the basic function of the subject matter at issue.

Various modifications and variations of the disclosed methods, compositions and uses of the invention will be apparent to the skilled person without departing from the scope and spirit of the invention. Although the subject matter has been disclosed in connection with specific preferred aspects or embodiments, it should be understood that the subject matter as claimed should not be unduly limited to such specific aspects or embodiments.

The subject matter may be implemented using hardware, software, or a combination thereof and may be implemented in one or more computer systems or other processing systems. In one aspect, the subject matter is directed toward one or more computer systems capable of carrying out the functionality described herein.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments±100%, in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

What is claimed is:

1. A couple-based digital reproductive health system, comprising:
a. a server, the server including a reproductive health application, ML algorithms, multimodal AI, reproductive health GUI, controller, operating memory, and a communications interface, wherein the server is accessible via a network;
b. a data store in communication with the server; and
c, wherein the controller is configured to execute stored program instructions, comprising:
i. collecting data related to a subject couple;
ii. analyzing the collected data related to the subject couple;
iii. creating a digital copy of the subject couple based on the analyzed collected data;
iv. generating personalized interventions and/or predictive outcomes based on the analyzed collected data related to the subject couple;
v. implementing the generated personalized interventions and/or predictive outcomes in the digital copy of the subject couple;
vi. presenting personalized interventions and/or predictive outcomes to the subject couple based on results from implementing the generated personalized interventions and/or predictive outcomes in the digital copy of the subject couple; and
vii. tracking results data of the presented personalized interventions and/or predictive outcomes implemented by the subject couple.

2. The system of claim 1, wherein the data related to the subject couple is collected via data modalities, and wherein the data modalities comprise exposome data and biological data.

3. The system of claim 2, wherein the biological data comprises omics data.

4. The system of claim 1, wherein the presented personalized interventions and/or predictive outcomes comprise the generated personalized interventions and/or predictive outcomes that resulted in a positive outcome when implemented in the digital copy of the subject couple.

5. The system of claim 1, wherein the multimodal AI and/or ML algorithms process the collected data related to the subject couple, create the digital copy of the subject couple, generate the personalized interventions and/or predictive outcomes, implement the generated personalized interventions and/or predictive outcomes in the digital copy of the subject couple, and/or track the results data of the presented personalized interventions and/or predictive outcomes implemented by the subject.

6. The system of claim 1, wherein the subject couple comprises one of a man and a woman, a woman and a sperm donor, or a man and an egg donor.

7. The system of claim 1, further comprising a reproductive family health archive, wherein the reproductive family health archive comprises an archive of data collected from each subject couple using the couple-based digital health platform system.

8. The system of claim 7, wherein the reproductive family health archive further comprises data collected from one or more offspring of the subject couple.

9. The system of claim 1, further comprising one or more entities in communication with the server via the network, wherein the one or more entities comprise healthcare providers, one or more laboratory service providers, one or more pharmacies, and/or one or more IVF providers.

10. The system of claim 1, wherein the instructions further comprise generating alternative personalized interventions and/or predictive outcomes for the subject couple.

11. The system of claim 10, wherein the multimodal AI and/or ML algorithms generate the alternative personalized interventions and/or predictive outcomes.

12. The system of claim 1, wherein the data store stores one or more of data modalities, subject couple health data, other entities data, personalized predictions data, personalized interventions data, and/or reproductive family health archive data.

13. A method of using a couple-based digital reproductive health platform, the method comprising:
a. providing a couple-based digital reproductive health system, comprising:
i. a server, the server including a reproductive health application, ML algorithms, multimodal AI, reproductive health GUI, controller, operating memory, and a communications interface, wherein the server is accessible via a network;
ii. a data store in communication with the server; and
iii. wherein the controller is configured to execute stored program instructions, comprising:
collecting data related to a subject couple;
analyzing the collected data related to the subject couple;
creating a digital copy of the subject couple based on the analyzed collected data;
generating personalized interventions and/or predictive outcomes based on the analyzed collected data related to the subject couple;
implementing the generated personalized interventions and/or predictive outcomes in the digital copy of the subject couple;
presenting personalized interventions and/or predictive outcomes to the subject couple based on results from implementing the generated personalized interventions and/or predictive outcomes in the digital copy of the subject couple; and
tracking results data of the presented personalized interventions and/or predictive outcomes implemented by the subject couple;
b. collecting data related to a subject couple;
c. analyzing the collected data related to the subject couple;
d. creating a digital copy of the subject couple based on the analyzed collected data;
e. generating personalized interventions and/or predictive outcomes based on the analyzed collected data related to the subject couple;
f. implementing the generated personalized interventions and/or predictive outcomes in the digital copy of the subject couple;
g. presenting personalized interventions and/or predictive outcomes to the subject couple based on results from implementing the generated personalized interventions and/or predictive outcomes in the digital copy of the subject couple; and
h. tracking results data of the presented personalized interventions and/or predictive outcomes implemented by the subject couple.

14. The method of claim 13, wherein the data related to the subject couple is collected via data modalities, and wherein the data modalities comprise exposome data and biological data.

15. The method of claim 14, wherein the biological data comprises omics data.

16. The method of claim 13, wherein the presented personalized interventions and/or predictive outcomes comprise the generated personalized interventions and/or predictive outcomes that resulted in a positive outcome when implemented in the digital copy of the subject couple.

17. The method of claim 13, wherein the multimodal AI and/or ML algorithms processes the collected data related to the subject couple, create the digital copy of the subject couple, generate the personalized interventions and/or predictive outcomes, implement the generated personalized interventions and/or predictive outcomes in the digital copy of the subject couple, and/or track the results data of the presented personalized interventions and/or predictive outcomes implemented by the subject.

18. The method of claim 13, wherein the subject couple comprises one of a man and a woman, a woman and a sperm donor, or a man and an egg donor.

19. The method of claim 13, further comprising a reproductive family health archive, wherein the reproductive family health archive comprises an archive of data collected from each subject couple using the couple-based digital health platform system.

20. The method of claim 19, wherein the reproductive family health archive further comprises data collected from one or more offspring of the subject couple.

21. The method of claim 13, further comprising one or more entities in communication with the server via the network, wherein the one or more entities comprise healthcare providers, one or more laboratory service providers, one or more pharmacies, and/or one or more IVF providers.

22. The method of claim 13, wherein the instructions further comprise generating alternative personalized interventions and/or predictive outcomes for the subject couple.

23. The method of claim 22, wherein the wherein the multimodal AI and/or ML algorithms generate the alternative personalized interventions and/or predictive outcomes.

24. The method of claim 13, wherein the data store stores one or more of data modalities, subject couple health data, other entities data, personalized predictions data, personalized interventions data, and/or reproductive family health archive data.
